SEQUENCE LISTING (2 sequences)

SEQ ID NO: 1 - DNA, 26 bp, Artificial Sequence
  PCR primer
tatgcatgagcacctggctgcggcgg

SEQ ID NO: 2 - DNA, 28 bp, Artificial Sequence
  PCR primer
ggccggcctcgacttcgtgatcgcctga

(19) United States
(12) Patent Application Publication
(10) Pub. No.: US 2004/0087003 A1
(43) Pub. Date: May 6, 2004

(54) Methods and cells for improved production of polyketides

(76) Inventors: Zhihao Hu, Castro Valley, CA (US); C. Richard Hutchinson, San Mateo, CA (US)

Correspondence Address: MORRISON & FOERSTER LLP, 755 PAGE MILL RD, PALO ALTO, CA, 94304-1018, US

(21) Appl. No.: 10/461,857

(22) Filed: Jun. 13, 2003

Related U.S. Application Data
(60) Provisional application No. 60/389,089, filed on Jun. 13, 2002.

(51) Int. Cl. C12N 1/21; C12P 19/60

(52) U.S. Cl. 435/252.33; 435/252.3; 435/75

(57) ABSTRACT

Described are host cells that contain a polyketide synthase gene and a thioesterase II gene, where the polyketide synthase gene has been modified to prevent utilization of its native starter unit for its expressed polyketide synthase. Also described are host cells containing a polyketide synthase gene and an endogenous thioesterase II gene, where the activity of the endogenous thioesterase II gene product has been decreased or eliminated. Methods for culturing these cells to produce polyketides are also provided, as are the polyketides produced.

### CROSS-REFERENCE TO RELATED APPLICATIONS

This application claims benefit under 35 U.S.C. §119(e) of provisional application no. 60/389,089, filed Jun. 13, 2002, which is hereby incorporated by reference herein in its entirety.

### TECHNICAL FIELD

The invention relates to cells that produce polyketides and methods of using them. The invention finds application in the fields of biomedicine, veterinary medicine, and agriculture.

### BACKGROUND OF THE INVENTION

Complex polyketides are a large family of bacterial natural products, many of which have considerable medicinal importance. Although complex polyketides have diverse structures (O'Hagan, 1993; full citations of cited references are found at the end of the specification), the carbon framework of these compounds is created by one type of enzyme called modular polyketide synthases (PKSs). Each of these large, multifunctional proteins, such as the ones responsible for the biosynthesis of erythromycin (Cortes et al., 1990; Donadio & Katz, 1992; Donadio et al., 1991), rapamycin (Aparicio et al., 1996; Molnar et al., 1996; Schwecke et al., 1995), rifamycin (August et al., 1998), and FR-008 (Hu et al., 1994), consists of sets of modules, and each module contains two or more enzymatic domains that catalyze a particular round of polyketide chain extension from simple acyl-coenzyme A (CoA) substrates.

6-deoxyerythronolide B (6dEB), the aglycone of the erythromycins, is synthesized by the 6-deoxyerythronolide synthase (DEBS) system, which consists of three large proteins: DEBS1, DEBS2 and DEBS3. The biosynthesis of 6dEB starts with the acyltransferase domain (AT_{L}) of the loading module selecting and loading propionyl-CoA onto the acyl carrier protein (ACP_{L}) in the same module. After the ACP_{L}-bound propionyl group is transferred to the first ketosynthase (KS1) active site of DEBS1, KS1 can catalyze the decarboxylative condensation between the propionate thioester, transferred onto KS1 of DEBS from the ACP_{L} of the didomain module, and a 2-methylmalonate thioester attached to the 4′-phosphopantetheinyl group of the ACP1 domain, which has been loaded by the AT1 domain of module 1. The resulting formation of a 2-methyl-3-ketopentanoyl-ACP1 thioester represents the typical reaction catalyzed by the three basic domains in a module. The intermediate product can be passed onto the KS domain in another module or, as in the case of module 1 of DEBS1, can be reduced by the ketoreductase (KR1) domain before its transfer. The other modules repeat the basic process step-by step, with or without reduction of the β-carbonyl, dehydration of the β-hydroxy and reduction of the α,β-C=C, in the manner dictated by the domain organization of modules and the architecture of DEBS. For the latter, the product 6dEB is synthesized from one propionyl-CoA starter unit and six methylmalonyl-CoA extender units through six rounds of decarboxylative condensation.

Studies have shown that selection of the primer and extender units is primarily controlled by the AT domains within each module (Donadio et al., 1991; Haydock et al., 1995; Lau et al., 2000; Marsden et al., 1994). Extender AT domains have a high degree of substrate specificity whereas the AT_{L} domain in DEBS1 exhibits considerable flexibility. Acetyl-CoA, butyryl-CoA and isobutyryl-CoA can serve as starter units in addition to propionyl-CoA in a heterologous host or in vitro (Kao et al., 1994; Lau et al., 2000; Wiesmann et al., 1995).

Small type II thioesterase (TEII) proteins are encoded by genes found in most modular PKS and non-ribosomal peptide synthetase (NRPS) gene clusters. The elimination of endogenous TEII greatly reduces the production of polyketide in some species, and the addition of a heterologous TEII can restore polyketide production. In other species, disruption of TEII gene does not lead to decreases in the production of polyketides. It has been proposed that TEII gene products play a role in removing aberrant groups attached by thioester linkage to ACP domains with the PKS extension modules, which might otherwise block the normal chain elongation process.

Recombinant host cells have been produced that contain heterologous PKS or endogenous PKS with modified modules. Such cells can be of species that normally produce polyketides as well as species that do not normally produce polyketides, and have been shown to produce novel polyketides or to produce known polyketides in greater amounts compared to non-recombinant cells. In view of the utility of polyketides as antibiotics and for other uses, there exists a need to produce known polyketides in greater amounts or to produce novel or unusual polyketides by further modifications of host cells.

### BRIEF SUMMARY OF THE INVENTION

In one aspect, the invention provides a host cell that contains a thioesterase II (TEII) gene and a mutated polyketide synthase (PKS) gene that produces an altered PKS incapable of using the native starter unit. The polyketide synthase may be altered such that the ketosynthase of module 1 is inactive, the loading module is deleted or inactivated, or in other ways that prevent incorporation of the native starter unit into the polyketide product. The PKS gene and the TEII gene may, independently, be endogenous or heterologous to the host cell. In one embodiment, the host cell contains a heterologous 6-deoxyerythronolide B synthase (DEBS) gene and a heterologous cognate thioesterase II gene, where the DEBS gene has been modified so that the ketosynthase catalytic domain of module 1 of the DEBS gene product is inactive. Exemplary host cells are S. erythraea, S. coelicolor, S. lividans, or E. coli.

In another aspect, the invention provides host cells that contain a heterologous PKS gene and an endogenous TEII gene, where the activity of the TEII gene product has been decreased or eliminated. In some embodiments, the activity of the TEII gene product has been decreased or eliminated due to a recombinant modification of the TEII gene of the host cell. In an embodiment, the host cell produces a polyketide that is not produced by the host cell before introduction of the heterologous PKS gene and decrease or elimination of the endogenous TEII activity. In a further embodiment, the invention encompasses host cells of the species S. erythraea that contain an endogenous or heterologous PKS gene or gene cluster and a native TEII gene, where the activity of the TEII gene product has been decreased or eliminated.

In one aspect of the invention, cells as described above are cultured under conditions such that a polyketide is produced and the polyketide is recovered. In some embodiments, the methods encompass culturing a host cell that contains a PKS gene that is modified to prevent utilization by the PKS of its normal starter unit and a TEII gene, and, optionally, recovering a polyketide that is produced. In some embodiments, the PKS gene is a 6-deoxyerythronolide B synthase gene. One embodiment includes culturing a host cell that contains a heterologous 6-deoxyerythronolide B synthase (DEBS) gene and a heterologous cognate thioesterase II gene, where the DEBS gene has been modified so that the ketosynthase catalytic domain of module 1 of the DEBS gene product is inactive and, optionally, recovering a polyketide that is produced. Exemplary host cells for this embodiment are S. erythraea, S. coelicolor, S. lividans, and E. coli. In some embodiments, the methods encompass culturing a host cell that contains a heterologous PKS gene and an endogenous TEII gene, where the activity of the endogenous TEII gene has been decreased or eliminated, so that the cultured host cells produce polyketides that are not produced by the host cell before transfection with the PKS gene and decrease or elimination of the endogenous TEII activity. An exemplary such polyketide is 15-nor-6-deoxyerythronolide B. In one of these embodiments, the host cell is S. erythraea.

### BRIEF DESCRIPTION OF THE DRAWINGS

FIG. 1(A) The model for 6dEB and 15-nor-6dEB production by the 6-deoxyerythronolide B synthase (DEBS) system. In heterologous hosts like S. coelicolor and S. lividans, both propionyl-CoA and acetyl-CoA are used by the PKS as starter units, whereas in the native host, S. erythraea, propionyl-CoA is preferentially used to produce the aglycone for the biosynthesis of the erythromycins. FIG. 1(B) Loading of the preferred substrate, propionyl-CoA, onto the ACP_{L} of DEBS does not invoke the editing function of the ery-ORF5 TEII enzyme whereas FIG. 1(C) non-specific loading of other acyl-CoA substrates results in hydrolysis of the acyl-ACP_{L} thioester by this enzyme.

FIG. 2(A) LC/MS data for the Streptomyces control (upper) and TEII⁺ (lower) strains and FIG. 2(B) LC/MS data for the S. erythraea control (upper) and ery-ORF5 TEII mutant (lower) strains.

FIG. 3(A) Southern hybridization of genomic DNA from the ery-ORF5 TEII mutant KOS146-171. KOS146-129c was used as the probe. Lane 1, genomic DNA from K41-135 strain digested with XhoI and BglII; lane 2, genomic DNA from KOS146-171 strain digested with XhoI and BglII; lane 3, genomic DNA from K41-135 strain digested with BglII; lane 4, genomic DNA from KOS146-171 strain digested with BglII; lane 5, genomic DNA from K41-135 strain digested with BamHI and BglII; and lane 6, genomic DNA from KOS146-171 strain digested with BamHI and BglII. FIG. 3(B) and FIG. 3(C) Genes, restriction enzyme sites and predicted size of restriction fragments surrounding the ery-ORF5 TEII genes in the KOS146-171 and K41-135 strains, respectively.

### DETAILED DESCRIPTION OF THE INVENTION

### 1. Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

As used herein, “host cell” means a cell, or progeny of a cell, that has been subject to recombinant modification and (1) produces, or is capable of producing, a polyketide not produced by an unmodified cell, or (2) has increased production of a polyketide that is produced by the unmodified cell. Examples of recombinant modification include the introduction into the cell of a heterologous polynucleotide and/or the inactivation of an endogenous gene.

As used herein, “heterologous” in reference to a PKS or TEII gene or protein in a recombinantly modified cell (or progeny of a recombinantly modified cell) means a gene or protein not found in an unmodified cell of the specified species or strain (e.g., a non-recombinant cell). One example of a heterologous gene is a gene from a first species that is introduced into a cell of a second species (e.g., by introduction of a recombinant polynucleotide encoding the gene). Another example of a heterologous gene is a gene (in a cell) that encodes a chimeric PKS.

As used herein, the “endogenous” PKS or TEII gene refers to the gene native to cells of the host species or strain. Endogenous genes that are recombinantly modified, e.g., genes comprising a KS1° mutation, are also considered endogenous genes.

As used herein, “polyketide synthase gene” refers generally to PKS genes encoding the portions of the core PKS (e.g., eryAI, eryAII, and eryAIII) and, optionally, other genes from the PKS gene cluster.

As used herein, two genes or proteins are “cognates” if both are found in the genome of the same species or strain of cell. For example, genes encoding a KS1 domain polypeptide and thioesterase II from Streptomyces venezuelae are cognates, but genes encoding a KS1 domain polypeptide from Streptomyces venezuelae and a thioesterase II from S. lividans are not.

The practice of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as Current Protocols in Molecular Biology (F. M. Ausubel et al., eds., 1987, including supplements through 2001); Molecular Cloning: A Laboratory Manual, third edition (Sambrook and Russel, 2001); PCR: The Polymerase Chain Reaction, (Mullis et al., eds., 1994); Current Protocols in Immunology (J. E. Coligan et al., eds., 1999, including supplements through 2001). Methods for the genetic manipulation of Streptomyces are described in Kieser et al, 2000, “Practical Streptomyces Genetics,” The John Innes Foundation, Norwich.

### 2. Introduction

In certain aspects, the invention provides host cells useful for production of polyketides, methods of producing polyketides using the host cells, and polyketides produced by the host cells.

In one aspect, host cells of the invention comprise a modified polyketide synthase (PKS) gene and a thioesterase II (TEII) gene, where the PKS gene has been modified to prevent utilization of the native starter unit for its expressed PKS. These cells allow production of polyketides with structures dependent on the non-native starter unit utilized by the modified PKS in quantities greater than in comparable cells without the TEII gene.

In another aspect, host cells of the invention comprise a modified endogenous TEII gene and a heterologous PKS gene, where the activity of TEII gene product has been reduced or abolished, either by modification or deletion of the TEII gene or by inhibition of the TEII enzyme. These host cells are useful for production of polyketides not normally produced by the cells. These host cells are also useful for production of polyketides that are produced by cells comprising an active TEII, but in greater quantities.

In related aspects, the invention provides methods of producing polyketides using host cells of the invention, and polyketides produced according to these methods.

Certain aspects of the invention are discussed in greater detail in the following sections. Section 3 describes polyketide synthases, section 4 describes type II thioesterases, section 5 describes host cells of the invention; and section 6 discusses certain methods of producing polyketides. It will be understood that this division into sections is for convenience and not limitation, and specific embodiments of the invention will comprise elements discussed on each of the sections.

### 3. Polyketide Synthases

Polyketides are synthesized in nature by polyketide synthase (PKS) enzymes. Two major types of PKS enzymes, differing in their composition and mode of synthesis, are known: the Type I or “modular” PKSs and Type II “iterative” PKSs. Modular PKSs produce polyketides by multistep pathways involving decarboxylative condensations between acyl thioesters followed by cycles of varying β-carbon processing activities and are responsible for producing a large number of 12-, 14-, and 16-membered macrolide antibiotics including erythromycin, megalomicin, methymycin, narbomycin, oleandomycin, picromycin, and tylosin. Each ORF of a modular PKS can comprise one, two, or more “modules” of ketosynthase activity, each module of which consists of at least two (if a loading module) and more typically three (for the simplest extender module) or more enzymatic activities or “domains.”

In one aspect, host cells of the invention include cells that contain a TEII-encoding gene and a PKS gene, where the latter is modified to prevent utilization of the native starter unit by its expressed PKS. The PKS gene can be endogenous to the host cell or heterologous to the host cell. Methods for modification of a PKS gene to prevent utilization of the native starter unit are well known and include those disclosed in, for example, published U.S. patent application Ser. No. 2003/0044938. Approaches to this modification of the PKS include inactivating the ketosynthase (KS), acyl transferase (AT) or acyl carrier protein (ACP) functions of module 1. A variety of methods are known for introducing mutations in PKS genes (e.g., site specific mutagenesis techniques). For example, one useful approach to modify the PKS to prevent utilization of native starter unit is to modify the KS activity in module 1 which results in the ability to incorporate alternative starter units as well as module 1 extender units.

Polyketide synthesis by cells comprising the modified PKS can be initiated by feeding chemically synthesized analogs of module 1 diketide products. The polyketides produced as a result of the modified PKS clusters will differ in the substituents that correspond to the residue of the starter unit in the finished polyketide. Exemplary starter units for use with a modified PKS (e.g., modified DEBS) are diketides, such as those disclosed in published U.S. patent application Ser. No. 2003/0044938. Methods to modify PKS enzymes to permit efficient incorporation of diketides are described in U.S. Pat. No. 6,080,555. In some embodiments the starter diketide is propyl diketide, (2S,3R)-3-hydroxy-2-methylhexanoate N-acylcysteamine thioester. Since the diketide intermediate is being supplied to the modified PKS cluster, the nature of the extender unit incorporated immediately adjacent the starter unit may also be varied.

Any of a variety of type II PKS can be used, e.g., in modified form, according to the invention. Nucleotide sequences for a multiplicity of PKSs are known and facilitate their use in recombinant procedures for producing a desired PKS product. For example, the nucleotide sequences for genes related to the production of erythromycin are disclosed in U.S. Pat. No. 6,004,787 and U.S. Pat. No. 5,998,194; for avermectin in U.S. Pat. No. 5,252,474; for FK506 in U.S. Pat. No. 5,622,866; for rifamycin in WO98/7868; for spiramycin in U.S. Pat. No. 5,098,837. These are merely examples. A more extensive listing, including a listing of tailoring enzymes found in PKS gene clusters, may be found in WO 01/27284 and in U.S. Pat. No. 6,524,841. Many others are known in the literature and available through, e.g., Genbank. In addition, methods to modify native PKS genes to alter the nature of the polyketide produced have been described. For example and not limitation, production of hybrid modular PKS proteins and synthesis systems is described and claimed in U.S. Pat. No. 5,962,290; methods to modify PKS enzymes by producing chimeric enzymes are described in U.S. Pat. No. 6,558,942; methods to alter the specificity of modules of modular PKSs to incorporate particular starter or extender units are described in U.S. Pat. No. 6,221,641; and improved methods to prepare diketides for incorporation into polyketides are described in U.S. Pat. No. 6,492,562. The polyketides produced in native hosts are generally subsequently tailored to obtain the finished antibiotic by oxidizing, hydroxylating, methylating, acylating, glycosylating, or otherwise modifying the product of the PKS or a modified polyketide. See, e.g., U.S. Pat. Nos. 6,403,775; 6,492,562; 6,399,789; and 5,998,194; and published U.S. patent application Ser. No. US 2002/0192767. The genes of the PKS, together with the tailoring genes and other genes related to polyketide synthesis and tailoring, constitute the PKS gene cluster.

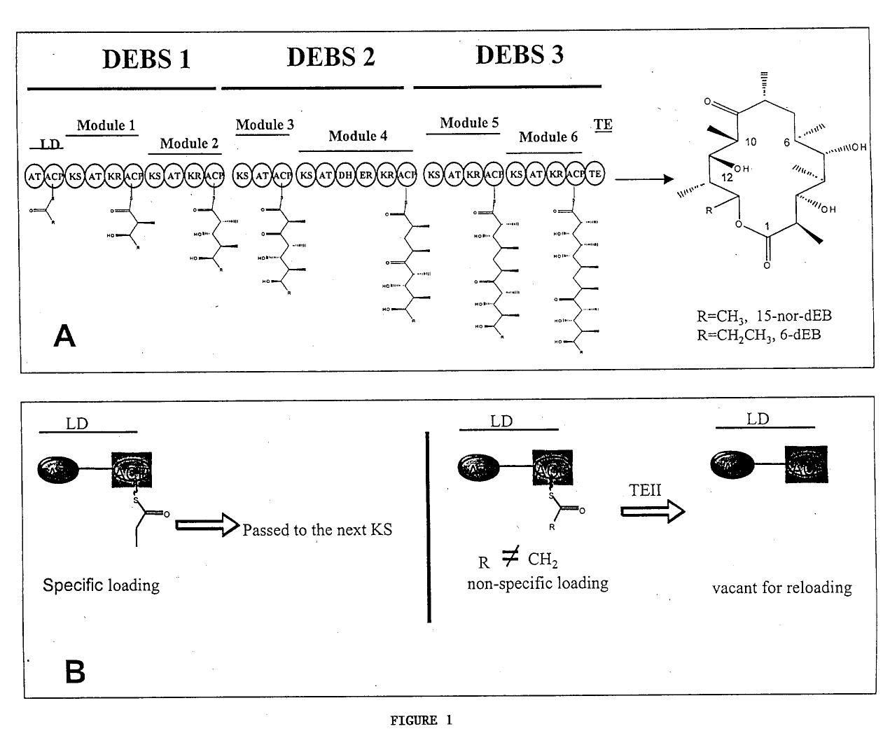

An exemplary PKS gene is that involved in biosynthesis of 6-deoxyerythronolide B (6-dEB), the macrocyclic core of the antibiotic erythromycin that is produced in Saccharopolyspora erythraea. 6-dEB and its derivatives constitute an important class of natural products. The PKS that results in the synthesis of 6-dEB is produced in S. erythraea. The 6dEB PKS gene and gene cluster from Sac. erythraea will be described in more detail to illustrate the general principles of PKS operation, but it will be understood that other PKS genes are also suitable for the invention. The 6-deoxyerythronolide synthase (DEBS) system is responsible for the biosynthesis of 6-deoxyerythronolide B (6dEB), the aglycone of the erythromycins, and consists of three large proteins—DEBS1, DEBS2 and DEBS3, encoded by the eryAI, eryAII, and eryAIII genes in Sac. erythraea (Caffrey et al., 1992; Cortes et al., 1990; Donadio et al., 1991) (FIG. 1). Other DEBS genes and proteins are found in other organisms, for example in Micromonospora megalomicea, as described in U.S. Pat. No. 6,524,841. The biosynthesis of 6dEB starts with the acyltransferase domain (AT_{L}) of the loading module selecting and loading propionyl-CoA onto the acyl carrier protein (ACP_{L}) in the same module. After the ACP_{L}-bound propionyl group is transferred to the first ketosynthase (KS1) active site of DEBS1, KS1 can catalyze the decarboxylative condensation between the propionate thioester, transferred onto KS1 of DEBS from the ACP_{L} of the didomain module, and a 2-methylmalonate thioester attached to the 4′-phosphopantetheinyl group of the ACP1 domain, which has been loaded by the AT1 domain of module 1. The resulting formation of a 2-methyl-3-ketopentanoyl-ACP1 thioester (FIG. 1(A), R=H) represents the typical reaction catalyzed by the three basic domains in a module. The intermediate product can be passed onto the KS domain in another module or, as in the case of module 1 of DEBS1, can be reduced by the ketoreductase (KR1) domain before its transfer (FIG. 1). The other modules repeat the basic process step-by step, with or without reduction of the β-carbonyl, dehydration of the β-hydroxy and reduction of the α,β-C=C, in the manner dictated by the domain organization of modules and the architecture of DEBS. For the latter, the product 6dEB is synthesized from one propionyl-CoA starter unit and six methylmalonyl-CoA extender units through six rounds of decarboxylative condensation.

### 4. Thioesterase II Genes

In addition to a PKS gene or gene cluster described above, the host cells of the invention generally also contain a TEII gene, which may be modified or unmodified, depending on the goals of the practitioner. In one aspect, the invention encompasses a host cell that contains a PKS gene (e.g., endogenous or heterologous) that is modified to prevent utilization of the native starter unit, and a functional TEII gene. In another aspect, the invention encompasses host cells that contain a heterologous PKS gene, where the activity of gene product of the endogenous TEII gene has been reduced or eliminated. In this aspect, the heterologous PKS usually is not modified to prevent utilization of the native starter unit for its expressed PKS, although it may comprise other modifications.

Thioesterases are involved in PKS and non-ribosomal polypeptide synthetase (NRPS) activity. Usually, two types of thioesterases are involved in PKS and NRPS function. A type I thioesterase (TE) domain is usually found at the carboxyl terminus of the last module to act in the sequence of events catalyzed by a PKS or NRPS, whereas the type II thioesterase (TEII) enzymes are separate, single proteins. The type I TE is responsible for release of the acyl-chain from the PKS (Gokhale et al., 1999), NRPS (Kohli et al., 2001; Schwarzer et al., 2001), or NRPS/PKS hybrid (Tang et al., 2000), whereas the exact mechanism of the TEII is presently not clear. It is believed that this enzyme plays an editing role by hydrolyzing incorrectly processed intermediates off the multifunctional PKS (Schwarzer et al., 2002). A study of model systems in vitro (Heathcote et al., 2001), supports the idea that the TEII enzyme associated with the tylosin PKS can remove aberrant short-chain fatty acids from ACP domains that have been mis-loaded with a fatty acid as a consequence of the erroneous decarboxylation of ACP-bound α-carboxythioesters. In more recent work the purified pikromycin pikAV TEII was shown to hydrolyze propionyl and butyryl derivatives of different ACP domains (including the ACP_{L} of DEBS1) about 3-fold faster than acetyl derivatives (Kim et al., 2002). Alternatively, the TEII may increase the intracellular activity of PKS enzymes by purging acyl carrier protein (ACP) domains that have been posttranslationally modified with an inappropriate phosphopantetheine donor (Butler, supra; and Heathcote, M. L., et al., Chemistry & Biology (2001) 8:207-220). Modifications of TEII activity are reported to have a variety of effects in cells. For example, co-expression of a cognate TEII gene with the pikromycin PKS genes increased production of both narbonolide and 10-deoxymethynolide in a Streptomyces host 2 to −7-fold, compared with the amount produced by a strain without the TEII gene (Tang et al., 1999). In the latter case and in other heterologous systems involving expression of the eryA DEBS genes in S. coelicolor or S. lividans, the macrolide products can still be produced in amounts greater than 160 mg l⁻¹ (in R6 medium) even in the absence of the cognate TEII gene. In contrast to these studies, TEII loss-of-function mutations in some bacterial PKS, NRPS and NRPS/PKS gene clusters have been reported to result in greatly reduced polyketide or oligopeptide production and the production of related antibiotics, including the following: tylosin (Butler et al., 1999), pikromycin (Xue et al, 1998), rifamycin (Doi-Katayama et al., 2000) and surfactin (Schneider & Marahiel, 1998).

A variety of TEII genes are known and may be used in embodiments in which the host cell contains a heterologous TEII gene. Non-limiting examples useful in the invention include the TEII genes of tylosin PKS of Streptomyces fradiae (Merson-Davies and Cundliffe (1994) Mol. Microbiol. 13: 349-355), pikromycin PKS of Streptomyces venezuelae (Xue et al. (1998) Proc. Natl. Acad. Sci. U.S.A. 95:12111-12116), rifarnycin PKS of Amycolatopsis mediterranei (August et al. (1998) Chem. Biol. (Lond.) 5:69-79), and the erythromycin PKS (DEBS) of Saccharopolyspora eythraea (Haydock et al. (1991) Mol. Gen. Genet. 230:120-8; see also Examples). TEII enzymes have also been found in bacterial NRPS, for example those that catalyze the production of macrocyclic peptide compounds (Schneider and Marahiel (1998) Arch. Microbiol. 169:404-10) and can be used in certain embodiments. Animal fatty acid synthases also express thioesterase (Witkowski et al. (1991) J. Biol. Chem. 266:18514-9).

Methods for cloning and heterologous expression of TEII genes in host cells are known in the art and these heterologous TEII genes and other TEII genes (or cells containing them) may be used in the present invention. For example, the TEII gene pikAV of the pikromycin biosynthetic gene cluster has been cloned (Kim et al., 2002, J. Biol. Chem. 277:48028-34). The nbmB TEII gene from S. Naronensis and the scoT gene from Streptomyces coelicolor have been heterologously expressed (Butler et al., 1999, Chem. Biol. (Lond.) 6:287-92; and (Kotowska et al., 2002, Microbiology 148:1777-83, respectively). In some embodiments of the invention a TEII gene from S. erythraea is used. The eryORF5 gene from the erythromycin biosynthetic gene cluster in S. erythraea encoding a TEII (Weber, J. M., et al., J. Bacteriol., 1990, 172:2372-2383) has been cloned under control of an IPTG-inducible T7 promoter on a chloramphenicol resistant plasmid, pGZ1 19EH (plasmid: Lessl, M., et al., J. Bacteriol., 1992, 174:2493-2500). This plasmid is compatible with plasmids pBP130 and pBP144, and has been used as a source of TEII in Streptomyces and E. coli. See, e.g., Pfeifer et al., 2002, Appl. Environ. Microbiol. 68: 3287-92, and published U.S. patent application Ser. No. 2002/0192767.

The activity of the gene product of an endogenous TEII in a host cell can be decreased or eliminated, when desired, using known methods. For example, the TEII gene can be modified to eliminate production of its gene product or to produce a gene product with reduced activity, or the TEII gene product can be inhibited. Methods are known for inactivation of TEII genes, for example, the rifR gene has been deleted from the rifamycin PKS gene cluster of A. mediterranei (Doi-Katayama et al., 2000, J. Antibiot. (Tokyo) 53:484-95), the tylO gene in the tylosin producer S. fradiae has been disrupted (Butler et al.,1999, Chem. Biol. (Lond.) 6:287-92), and the pikAV (Chen et al., 2002, Gene 263:255-64) and srfA-TE (Cosmina et al.,1993, Mol. Microbiol. 8:821-31) genes of the pikramycin PKS and NRPS, respectively, have been disrupted.

Alternatively, the gene product of TEII may be inhibited. By “inhibited” is meant partial or complete elimination of the catalytic activity of the TEII enzyme. Methods of enzyme inhibition are known in the art, and include competitive and noncompetitive inhibition. The latter includes irreversible inhibition, which may cause the activity of the enzyme to decrease to zero.

It may be desirable in some embodiments to reduce, but not completely eliminate, the activity of a TEII. Thus, modifications of the endogenous TEII gene to produce a TEII with various levels of decreased activity (e.g., by site-directed mutagenesis) may be used in some embodiments of the invention (see, e.g., Witkowski et al., 1991, J. Biol. Chem. 266:18514-19; Witkowski et al., 1992, J. Biol. Chem. 267:18488-92). Alternatively, if an inhibitor of TEII is used, its level may be calibrated to produce the desired decrease in TEII activity.

### 5. Host Cells

The invention provides host cells that contain and express the PKS and TEII genes described above. In one aspect, host cells of the invention include cells that contain a polyketide synthase (PKS) gene and a thioesterase II (TEII) gene, where the PKS gene has been modified to prevent utilization of the native starter unit for its expressed PKS. The PKS gene and the TEII gene may be, independently, endogenous to the host cell or heterologous to the host cell. In another aspect, host cells of the invention include cells containing a PKS gene, wherein the activity of the endogenous TEII gene product of said host cell has been decreased or eliminated.

Suitable host cells are prepared from cells that ordinarily produce polyketides, as well as cells that do not ordinarily produce polyketides. Microorganism hosts suitable for the synthesis of polyketides according to the invention include various strains of Streptomyces, in particular S. erythraea, S. coelicolor and S. lividans, various strains of Myxococcus, industrially favorable hosts such as Escherichia, preferably E. coli, Bacillus, Pseudomonas or Flavobacterium, Saccharopolyspora, and other microorganisms such as yeasts, such as Saccharomyces. Mammalian host cells can also be used.

When the modified PKS is heterologous to the host cell, the selected host may be modified to include any one of many possible polyketide synthase genes and their gene products by incorporating therein appropriate expression systems for the proteins included in such synthases. Either complete synthases or partial synthases may be supplied depending on the product desired. If the host produces polyketide synthase natively, and a different polyketide from that ordinarily produced is desired, it may be desirable to delete the genes encoding the endogenous PKS. Methods for such deletion are described in U.S. Pat. No. 5,830,750.

The host cells may additionally include genes in the PKS gene cluster that produce starter units and extender units, and that tailor the polyketide produced by the PKS by hydroxylation, glycosylation, and the like. Such genes are further described in published U.S. patent application Ser. No. US 2002/0192767. When the modified PKS is heterologous to the host cell, it is sometimes advantageous to supply the host cell with recombinant genes that code for proteins involved in the posttranslational modification of the PKS to its active state. When the modified PKS is endogenous to the host cell it is sometimes advantageous to disable one or more endogenous PKS(s) of the host cell. See, e.g., U.S. Pat. No. 5,672,491. It will be appreciated that, generally, the host cells of the invention include those genes (e.g., including the PKS core genes and others from the PKS gene cluster) required for biosynthesis of a polyketide under suitable culture conditions.

Streptomyces is a convenient host for expressing polyketides, because polyketides are naturally produced in certain Streptomyces species, and Streptomyces cells generally produce the precursors needed to form the desired polyketide. In some embodiments, the invention provides a recombinant Streptomyces host cell that expresses a recombinant PKS and/or TEII. In other embodiments, the invention provides a Streptomyces host cell where the activity of the endogenous TEII gene product of said host cell has been decreased or eliminated. In some of these embodiments the host cell further includes a heterologous PKS.

As noted supra, if a Streptomyces or other host cell ordinarily produces polyketides, it may be desirable to modify the host so as to prevent the production of endogenous polyketides prior to its use to express a recombinant PKS and/or TE II of the invention. Such modified hosts include S. coelicolor CH999 and similarly modified S. lividans described in U.S. Pat. No. 5,672,491, and PCT Publication Nos. WO 95/08548 and WO 96/40968. In such hosts, it may not be advantageous to provide enzymatic activities for all of the desired posttranslational modifications of the enzymes that make up the recombinantly produced PKS, because the host naturally expresses such enzymes. In particular, these hosts generally contain holo-ACP synthases that provide the phosphopantotheinyl residue needed for functionality of the PKS.

Host cells of the invention may be produced using genetic engineering techniques known in the art. If a heterologous PKS synthase is to be used, techniques known in the art may be used to clone and introduce the genes into the host cells. Similarly, genetic engineering techniques known to those of skill in the art may be used to clone and introduce TEII genes into cells. See, e.g., published U.S. patent application Ser. No. 2002/0192767.

Without limitation, the invention provides host cells that contain a heterologous polyketide synthase gene, modified to prevent utilization of native starter unit, and an endogenous TEII; host cells that contain an endogenous polyketide synthase gene, modified to prevent utilization of native starter unit, and an endogenous TEII; host cells that contain a heterologous polyketide synthase gene, modified to prevent utilization of native starter unit, and a heterologous TEII; and host cells that contain an endogenous polyketide synthase gene, modified to prevent utilization of native starter unit, and a heterologous TEII. In some embodiments, the host cell is selected from Streptomyces (e.g., S. coelicolor) and Escherichia (e.g., E. coli).

In some of the above embodiments, the PKS is modified to prevent utilization of the native starter unit through inactivation of the KS1 domain. In some embodiments, the host cell contains a heterologous 6-deoxyerythronolide B synthase (DEBS) gene and a heterologous cognate TEII gene, where the DEBS gene has been modified by inactivating the ketosynthase (KS) catalytic domain of module 1, and where the host cell is selected from the group consisting of S. erythraea, S. coelicolor, S. lividans, and E. coli. In some of these embodiments, the host cell is S. coelicolor. In some of these embodiments the host cell is E. coli.

Further embodiments of the host cells of the invention include those in which the activity of the TEII of the host cell has been decreased or eliminated, and the cell contains a heterologous PKS. In some embodiments, the activity of the host cell's TEII is eliminated by inactivation of the TEII gene, and the cells contain a heterologous PKS. In some of these embodiments, the host cell is S. erythraea and the PKS is DEBS. In other embodiments, the host cell is S. erythraea, the PKS is endogenous to the host cell, and the TEII of the host cell is eliminated by inactivation of the TEII gene.

In host cells such as yeasts, plants, or mammalian cells which ordinarily do not produce polyketides, it is advantageous to provide, also typically by recombinant means, enzymatic activity for posttranslational modification of the enzymes that make up the recombinantly produced polyketide synthase. For example, holo-ACP synthases may be provided (see WO 97/13845 and 98/27203). In hosts that normally produce polyketides, it may not be necessary to provide enzymatic activity for posttranslational modification of PKS enzymes.

It may be also advantageous to supply the host cell with one or more enzymes to produce starter and/or extender units for a PKS, typically including those that catalyze the conversion of the free acid to the CoA derivative, and/or if the foregoing enzymes are produced in a host, tailoring enzymes to activate them. In addition, it may be desirable to disarm catabolic enzymes which would otherwise destroy the appropriate starting materials. The above are described more fully in published U.S. patent application Ser. No. 2002/0192767.

### 6. Methods

The invention also provides methods of producing polyketides by culturing the host cells described above, and polyketides produced by the culture of the host cells.

Methods of culturing host cells for the production of polyketides of the invention are known in the art. See, e.g., published U.S. patent application Ser. No. 2002/0192767. Specific culture conditions for species of host cells useful in the invention are known in the art. Generally, the yield of polyketide is markedly increased by 1) maintaining relatively steady nutrient levels throughout the fermentation; 2) batch feeding additional precursor for starter and/or extender units, especially in the case of cells that partially or completely lack the ability to produce required quantities of starter or extender units; and 3) permitting growth to high cell densities.

As described above, when culturing host cells containing a PKS that has been modified to be incapable of utilizing native starter units, a supply of starter unit that may be used by the modified PKS is advantageous. Thus, in embodiments of the invention where the PKS is modified by inactivation of the KS1 domain (KS1° PKS), exogenous diketide is advantageously provided to the cells, as described above and in published U.S. patent application Ser. No. 2002/0192767. For example, when the modified PKS is DEBS the diketide supplied can be propyl diketide. This results in increased production of the polyketide 15-methyl-6dEB in Streptomyces hosts (see Examples). Exemplary methods include methods of culturing a host cell where the host cell contains a PKS gene that has been modified to prevent utilization of the native starter unit for the PKS, and a TEII gene; in one embodiment, the methods include culturing a host cell contains a heterologous 6-deoxyerythronolide B synthase gene and a heterologous cognate TEII gene, where the 6-deoxyerythronolide B synthase gene has been modified by inactivating the ketosynthase (KS) catalytic domain of module 1, and where the host cell is selected from the group consisting of S. erythraea, S. coelicolor, S. lividans, and E. coli. In some of these embodiments, the host cell is S. coelicolor. In some of these embodiments, the host cell is S. lividans. In some of these embodiments, the host cell is S. erythraea. In some of these embodiments, the host cell is E. coli.

The methods of the invention may further encompass recovering the polyketide or polyketides produced by the host cells. Methods of recovery of polyketides are known in the art, and include, for example, chromatographic methods.

The invention also provides polyketides produced by culturing the host cells. Exemplary polyketides of the invention include, but are not limited to, those produced by culturing Streptomyces spp. or Escherichia spp. modified to contain a inactivated TEII, such as isomers of 15-nor-6-deoxyerythromycin or of 15-nor-6-deoxyerythromycin. Non-limiting examples of species useful in producing polyketides of the invention include S. coelicolor, S. lividans, S. erythraea, and E. coli.

A detailed description of the invention having been provided above, the following examples are given for the purpose of illustrating the invention and shall not be construed as being a limitation on the scope of the invention or the claims. The contents of all references, patents and published patent applications cited throughout this application are incorporated herein by reference in their entirety.

### 7. EXAMPLES

### Example 1

This Example describes materials and methods used in Examples 2-5.

Strains and plasmids: Strains and plasmids made and used in this study are listed in Table 1.

Media and chemicals: R5 (Kieser, 2000) and Luria Broth (LB) solid medium were used for protoplast generation and strain propagation. TSB (Kieser, 2000), and R6 liquid medium (see below) was used for seed and fermentation media for Streptomyces strains. IT plate medium (see below) was used to maintain K41-135 and its derivatives. R6 medium is composed of (per liter) 103 g sucrose, 0.25 g K₂SO₄, 10.12 g MgCl₂.6H₂O, 0.96 g sodium propionate, 0.1 g Difco casaminoacids, 5.0 g yeast extract, 28.2 g Bis-Tris propane (Sigma) and 2.0 ml of trace elements solution (the same as used in the R5 medium). After autoclaving, the following aqueous solutions were added (per liter): 10 ml of 0.5% (w/v) KH₂PO₄, 8 ml of 2.5M CaCl₂.H₂O, and 15 ml of 20% L-proline. IT plate medium contains (per liter) 5 g of anhydrous glucose, 5 g of tryptone, 0.5 g of betaine hydrochloride, 5 g of starch, 1 g of corn steep liquor (50%), 200 mg of MgSO₂.7H₂O, 2 mg of ZnSO₄.7H₂O, 0.8 mg of CuSO₄.5H₂O, 0.2 mg of CoCl₂.6H₂O, 4 mg of FeSO₄.7H₂O, 80 mg of CaCl₂.6H₂O, 10 g of NaCl, 150 mg of KH₂PO4, and 20 g of agar, adjusted to pH7 by 20% NaOH.

For the E. coli Examples, antibiotics at the following concentrations were added to LB media: carbenicillin (carb) 100 μgml⁻¹, streptomycin (strep) 20 μgml⁻¹, and tetracycline (tet) 7.5 μgml⁻¹. E. coli fermentation media was supplemented with 5 mM sodium propionate, 50 mM monosodium glutamate and 50 mM succinic acid purchased from Sigma and prepared as stock solutions adjusted to pH 7.0.

### Example 2

Co-expression of ery-ORF5TEII with eryA DEBS genes in Streptomyces strains.

This example demonstrates increased yeild of polyketides in host cells that were contransformed with PKS and TEII.

For expression in Streptomyces strains the ery-ORF5 clone was prepared as follows. Using cosmid pKOS79-170 DNA (see Table 1) as the template, the ery-ORF5 gene was amplified by the PCR with the forward primer, [5′-d(TATGCATGAGCACCTGGCT GCGGGCGG)], [SEQ ID NO: 1] designed to introduce a NsiI site overlapping the start codon, and the reverse primer, [5′-d(GGCCGGCCTCGACTTCGTGATCGCCTGA)], [SEQ ID NO: 2] designed to introduce a NsiI site downstream of the stop codon (the restriction sites are shown in bold type). The PCR product was cloned into ZERO-Blunt (Invitrogen) then a 0.7 kb NsiI-NsiI (one NsiI site is from the vector) fragment containing the ery-ORF5 gene was transferred into NsiI cut pKOS146-83A (Table 1) to give plasmid pKOS146-101A. pKOS 146-83A was made from pUC119 (Vicira & Messing, 1987), in which the HindIII-EcoRI polylinker was replaced by a HindIII-EcoRI fragment containing the actII-ORF4 gene and the divergent actI and actIII promoters from pWHM467 (Wohlert et al., 2001). Three fragments, EcoRI-PacI fragment of pKOS146-101A, HindIII-PacI fragment of pKOS146-88A (Table 1), and EcoRI-HindIII fragment of pKOS146-87B (Table 1) were ligated together and packaged using a GigapackIII-plus (Stratagene) in vitro packaging kit. pKOS146-103A, identified from carbenicillin resistant E. coli transformants infected by the packaged mixture, contains the eryA DEBS genes and ery-ORF5 TEII gene under control of the actI and actIII promoters, respectively. pKOS 146-103A and pKOS146-109 were introduced by transformation into the S. lividans K4-114 and S. coelicolor CH999 strains separately. The titers of erythromycin aglycones were measured by HPLC/MS analysis of culture extracts, as described below.

Production and quantitation of 6-dEB and its analogs produced by Streptomyces strains. Streptomyces transformants were picked into 6 ml of TSB liquid medium with 50 mg l⁻¹ of thiostrepton and grown in culture tubes with shaking (250 rpm) at 30° C. After sufficient growth (normally 3-4 days), 2 ml portions of the cultures were transferred to 250 ml Erlenmeyer flasks containing 40 ml of R6 medium (supplemented with appropriate antibiotics, and in the case of strains containing the DEBS KS1° expression plasmid, the propyl diketide was fed at a final concentration of 1 g l⁻¹). The flasks were shaken at 30° C. and 250 rpm for about 7 days, after which 1 ml of culture was withdrawn and centrifuged (1,200×g, 5 min). Samples of the supernatants were analyzed by on-line extraction by LC-MS using a system comprised of a 10 port, 2 position switching valve/injector, Beckman System Gold high performance liquid chromatograph (HPLC), an Alltech evaporative light scattering detector (ELSD), and a PE-SCIEX API100LC mass spectrum (MS)-based detector configured with an atmospheric pressure chemical ionization source. Clarified whole broth (50 or 100 μL) was loaded onto a Metachem Metaguard Inertsil ODS-3 guard column (5 mm, 4.6×30 mm) that had been pre-equilibrated for 1 min with H₂O (0.1% HOAc) at 1 ml/min, with the eluate being diverted to waste. At 30 sec post-injection, a linear gradient to 15% MeCN (0.1% HOAc) over 1 min was initiated. At 2 min the direction of flow through the guard column was reversed, and the eluate was diverted onto a Metachem Inertsil ODS-3 column (5 mm, 4.6×150 mm) pre-equilibrated with 15% MeCN (0.1% HOAc). Compounds were eluted using a linear gradient from 15 to 100% MeCN (0.1% HOAc) at 1 ml min⁻¹ over 6 min, then 100% MeCN (0.1% HOAc) for 3 min. The eluate stream was split equally between the ELSD and MS detectors. Under these conditions, 6dEB elutes at 9.9 min and 15-nor-6dEB at 9.3 min. Titers were determined by comparing the ELSD response of samples to a standard curve constructed from a power fit to 15-nor-6dEB standards.

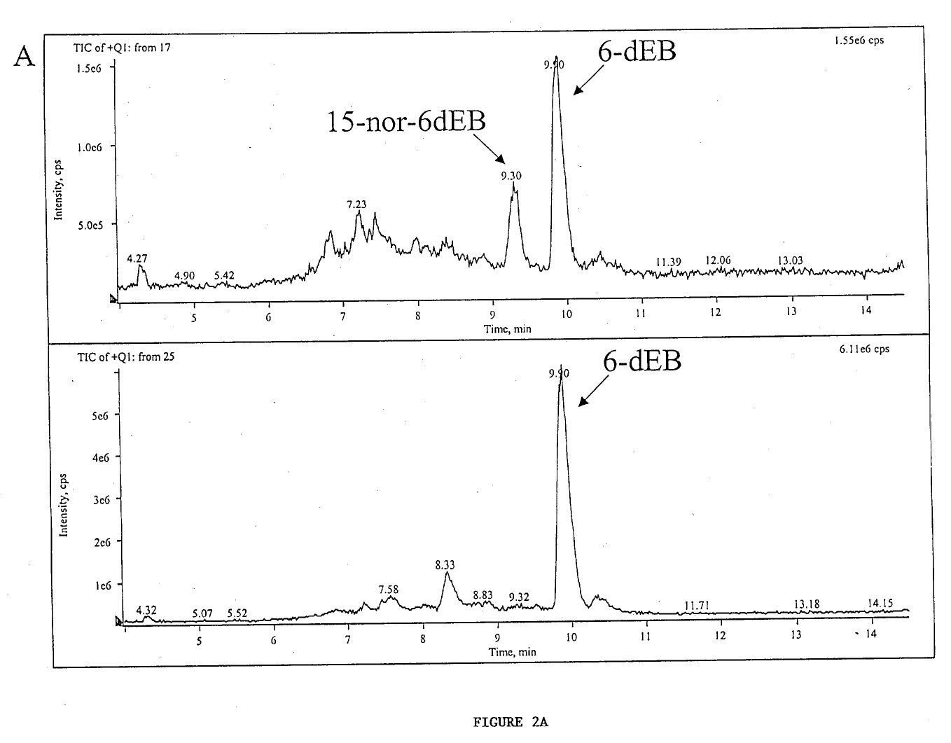

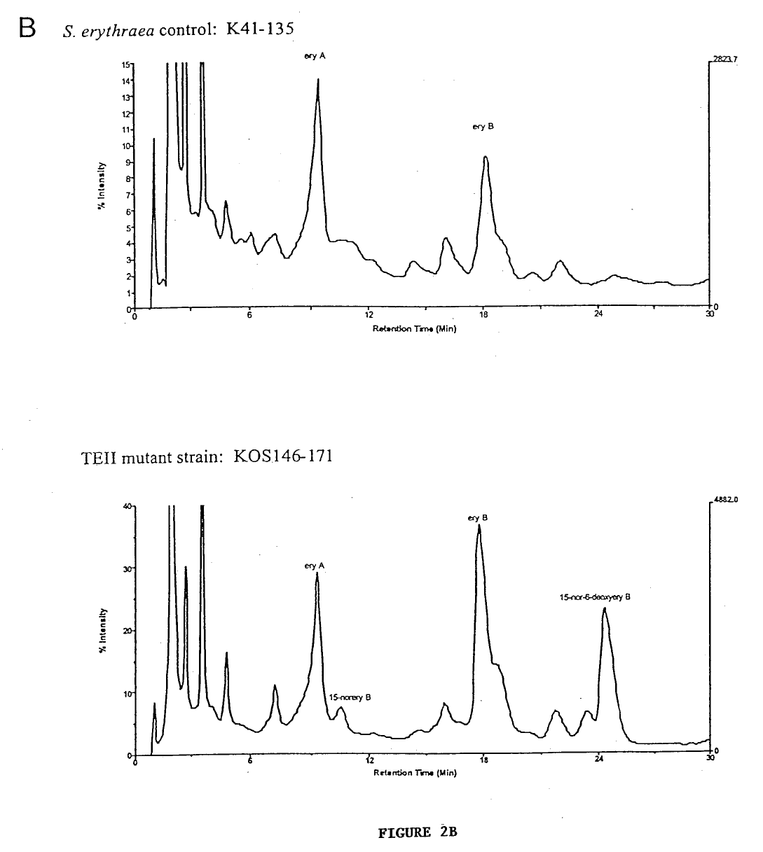

Co-expression of the TEII and DEBS genes was achieved by placing each of them under the control of the divergently oriented actIII and actI promoters, respectively, on a pRM5-derived vector (McDaniel et al., 1993) (see above) where the promoters are regulated by the positively acting actII-ORF4 gene to ensure expression in the early stationary stage of growth. Plasmid pKOS 146-103A (Table 1) carrying the ery-ORF5 TEII and DEBS genes was introduced by transformation into Streptomyces lividans K4-114 (Ziermami & Betlach, 1999) and S. coelicolor CH999 (Kao et al., 1994). In each host the combined yield of 6dEB and 15-nor-6dEB in the R6 growth medium, which contains 0.96 g l−1 of propionate but no glucose, was increased approx. 2-fold over the amount produced by a comparable strain without the ery-ORF5 TEII gene (75-89 mg l−1 vs. 30-40 mg l⁻¹). Only a trace amount of 15-nor-6dEB was produced (less than 2 mg l−1), whereas the ratio of 6dEB to 15-nor-6dEB produced in the same medium by the strain without the TEII gene was 2:1, as shown in FIG. 2(A). In the FM6-1 medium, which is not supplemented with propionate, 77.6% of the total polyketide product was 6dEB in the extract from the CH999/pKOS 146-103A strain tested, whereas only 33% was 6dEB in the extract from the CH999/pKOS11-26* (Table 1) control strain.

This Example shows that co-expression of the TEII gene approximately doubles the 6dEB titer and considerably lowers the amount of 15-nor-6dEB produced relative to 6dEB, even though the presence of this gene does not eliminate 15-nor-6dEB production as it does in a S. erythraea strain.

### Example 3

Co-expression of the ery-0RF5 TEII and eryA DEBS KS1° genes in Streptomyces hosts and polyketide production.

The host cells of this Example were constructed in the same manner as those of Example 2, except replacement of the BglII-PacI fragment of pKOS146-103A with the BglII-PacI fragment of pJRJ2 (Jacobsen et al., 1997) gave pKOS146-109, a DEBS KS1° version of pKOS146-103A. pKOS146-103A and pKOS146-109 were introduced by transformation into the S. lividans K4-114 and S. coelicolor CH999 strains separately. The titers of erythromycin aglycones were measured by HPLC/MS analysis of culture extracts, as described in Example 2.

The increased production of 6dEB in the presence of the TEII gene observed in Example 2 might have resulted from the decreased formation of 15-nor-6dEB due to an effect of TEII on the loading module only, an increased DEBS productivity due to the editing effect of TEII on other modules, or a combination of both types of activity. To study these possibilities, in Example 3 the TEII gene was co-expressed with the DEBS KS1° mutant that produces 15-methyl-6dEB when the racemic (2S, 3R)-2-methyl-3-hydroxylhexanoate N-propionyl cysteamine thioester (“propyl diketide”) is fed to the culture. In this case, the substrate was loaded onto the KS2 domain, bypassing both the loading module and module 1 of DEBS (Jacobsen et al., 1997). The pKOS146-109 plasmid (Table 1) was constructed from the DEBS KS1° genes in the same manner as pKOS146-103A and introduced into the K4-114 and CH999 strains by transformation.

Twice as much 15-methyl-6dEB (30-40 mg l⁻¹) was produced in the R6 medium by both the K4-114/pKOS146-109 and CH999/pKOS146-109 strains compared with the amount produced by control strains carrying pJRJ2 that lacks the TEII gene. This result shows that the positive effect of TEII on polyketide production can be due to more than just its effect on the loading module in these host strains.

The observation that expression of the ery-ORF5 TEII gene in Streptomyces hosts boosted the production of 6dEB and its analogs at least two-fold has been confirmed in E. coli (Pfeifer et al., 2002). Yet, in both types of bacteria studied here, the ery-ORF5 TEII gene clearly is not essential for DEBS to function properly, in contrast to the situation in some other actinomycetes where TEII mutations have caused nearly complete loss of macrolide biosynthesis (Butler et al., 1999; Doi-Katayama et al., 2000; Xue et al., 1998).

### Example 4

This Example demonstrates that a S. erythraea strain bearing a disrupted ery-ORF5 TEII gene produced a considerable amount of 15-norerythromycins, which were not found in culture extracts of the parent strain.

Construction of the S. erythraea ery-ORF5TEII mutant strain and polyketide production.

This mutant was obtained by gene disruption as follows. A BamH-BglII fragment (3 kb in size) containing eryF, ery-ORF5 and eryG genes from cosmid pKOS79-170 (Table 1) was subcloned into pLitmus 28 (BioLabs) previously cut with BamHI to make pKOS146-119. The kanamycin resistance gene (kan) from Supercos1 (Stratagene) was removed as a SmaI-StuI fragment and inserted into the PshAI site of pKOS146-119 to give pKOS146-129B. Then the XbaI-EcoRI fragment from pKOS146-129B containing eryF, ery-ORF5, eryG and the kan genes was ligated with pKOS97-49B, previously digested with SpeI and EcoRI, to make pKOS146-129C.

To construct the ery-ORF5 disruptant strain, plasmid pKOS146-129C was introduced by transformation into E. coli ET12567/pUB307 (Flett et al., 1997), then mobilized into the S. erythraea K41-135 strain by conjugation from the ET12567 transformants, selecting for apramycin resistant colonies on R5 plates (60 μgml⁻¹ of apraimycin). After sporulation and propagation of the apramycin resistant exconjugants of the K41-135/pKOS 146-129C recombinant strain on IT medium plates containing 50 μgml⁻¹ of kanamycin, kanamycin resistant, apramycin sensitive clones were chosen as potential double-crossover recombinants. The desired ery-ORF5 TEII disruptant strains were verified by Southern-blot hybridization against pKOS146-129C as explained in the text.

Production and quantitation of erythromycins and analogs produced by the S. erythraea ery-ORF5 TEII mutant. The S. erythraea K41-135 parental and ery-ORF5 mutant strains were cultured in TSB for about 3 days with shaking (250 rpm) at 34° C., then 2 ml of the culture was transferred into 40 ml of F1 medium (Brunker et al., 1998) in 250 ml flasks and fermentation was continued for 9 days Titers of erythromycins were determined as described elsewhere (Carreras et al., 2002).

Authentic standards of 15-norerythromycins were prepared by bioconversion of 15-nor-6-dEB using the methods described by Carreras et al. (2002). Authentic standards of 15-nor-6-deoxyerythromycins were prepared by the same methods using a mutant strain of S. erythraea having a defective eryF gene encoding the C6-hydroxylase. The fermentations were performed under the conditions described above, after which 1.5 ml of culture was withdrawn and centrifuged (1,200×g, 5 min). Samples of the supernatants were analyzed by on-line extraction by LC-MS. For LC-MS analyses, 20 μL of clarified broth was chromatographed on a Phenomenex Develosil column (5 mm, 4.6×150 mm) with a mobile phase of 39% 3:2 MeCN-EtOH (5 mM NH₄OAc) at 1 ml min⁻¹. The eluate was split 1:1 between a Sedex 55 ELSD and an Applied Biosystems Mariner time-of-flight mass spectrometer equipped with a turbo-ion spray source (source temp. 400° C.; spray tip potential 5500 V; nozzle potential 175 V). Under these conditions, standards of erythromycins C, A, D, and B eluted at 5.8, 9.3, 10.1, and 17.8 min, respectively. For exact mass measurements by LC-MS, the [M+H]⁺ and [M-C₈H₁₄O₃]⁺ ions of erythromycin B in samples were used to calibrate the mass scale.

The 15-nor-6-deoxyerythromycin B and 15-norerythromycin B were characterized by NMR (¹H, ¹³C, COSY, HSQC, and HMBC) and MS analyses: 15-nor-6-deoxyerythromycin B: ¹³C-NMR (CDCl₃, 100 MHz): δ 217.6 (C9),177.0 (C1), 104.3 (C1′), 97.0 (C1″), 84.0 (C5), 79.2 (C3), 78.0 (C4″), 72.5 (C3″), 70.6 (C2), 70.4 (C13), 70.3 (C11), 69.2 (C5′), 65.6 (C3′), 65.6 (C5″), 49.3 (3″OMe), 45.3 (C8), 44,7 (C2), 43.4 (C4), 41.9 (C12), 41.1 (C10), 40.3 (NMe₂), 35.7 (C6), 35.1 (C2″), 34.2 (C7), 28.5 (C4′), 21.4 (C6′), 21.2 (C3″Me), 19.6 (Me6), 18.2 (C6″), 18.0 (C14), 16.7 (Me8), 14.2 (Me2), 9.4 (Me4), 8.7 (Me12), 7.9 (Me10). HRMS: calcd for C₃₆H₆₆NO₁₁, 688.4630; found 688.4591.

15-norerythromycin B: ¹³C—NMR (CDCl₃, 100 MHz): δ 219.3 (C9), 176.0 (C1), 103.3 (C1′), 96.8 (C1″), 83.4 (C5), 78.0 (C3), 77.9 (C4″), 75.6 (C6), 72.6 (C3″), 70.8 (C2), 69.8 (C13), 69.7 (C11), 69.1 (C5′), 65.7 (C3′), 65.5 (C5″), 49.5 (3″OMe), 45.0 (C8), 44.7 (C2), 41.1 (C12), 40.3 (NMe₂), 40.3 (C4), 39.6 (C10), 38.4 (C7), 35.0 (C2″), 28.6 (C4′), 27.3 (Me6), 21.4 (C6′), 21.4 (Me3″), 18.6 (Me8), 18.5 (C6″), 18.2 (C14), 14.7 (Me2), 9.4 (Me4), 9.1 (Me10), 8.8 (Me12). HRMS: calcd for C₃₆H₆₆NO₁₂, 704.4580; found 704.4565.

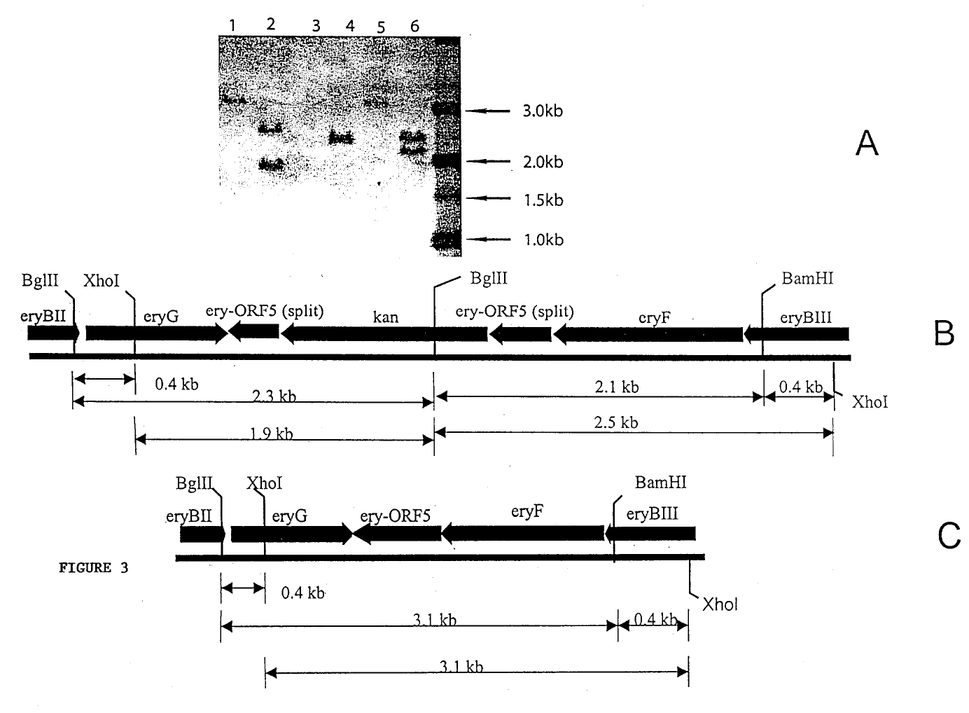

To ascertain whether the effect of the ery-ORF5 TEII gene noted above was indicative of its role in the normal host, we carried out a gene replacement experiment targeted at the chromosomal ery-ORF5 gene in an erythromycin-producing strain of S. erythraea. The kanamycin resistance gene (from Tn5) was inserted into the PshAI site of ery-ORF5 in place of the 3.1 kb BamHI-BglII segment, as described above, to give pKOS146-129C (Table 1). This plasmid was introduced into the S. erythraea K41-135 strain by interspecies conjugation and the transformants resistant to both kanamycin and apramycin were serially transferred in solid media without selection to isolate kanamycin resistant, apramycin sensitive strains. Southern analysis of genomic DNA isolated from such strains was used to identify the ery-ORF5 mutants. pKOS146-129C hybridized to 3.1 kb fragments in BglII+BamHI and XhoI+BglII digested DNA from the K41-135 strain (FIG. 3(A), lanes 5 and 1). Hybridization to a BglII fragment larger than 4 kb was seen also (data not shown). In a strain with the mutated ery-ORF5 gene, this probe hybridized to 2.3 kb and 2.1 kb fragments in BglII+BamHI digested DNA, 1.9 kb and 2.5 kb fragments in XhoI+BglII digested DNA, and a 2.3 kb fragment in BglII digested DNA (FIG. 3(A), lanes 6, 2 and 3). These data, when analyzed as shown in FIGS. 3(B) and 3(C), indicated that the TEII gene had been disrupted in the KOS 146-171 strain (Table 1) by insertion of the kanamycin resistance gene.

Fermentation of the KOS146-171 TEII mutant strain in the F1 medium optimized for erythromycin-production followed by LC-MS analysis of the products recovered in the culture extract showed that the mutant strain produced the expected mixture of the erythromycins, along with notable amounts of three 15-norerythromycins that were not seen in the extract of the K41-135 parental strain that had been carried through the same procedures (FIG. 2(B) shows the relative mobility of two of these three compounds). Elemental compositions of the three new compounds were determined by high resolution mass spectrometry (HRMS). Under the electrospray ionization conditions used, erythromycins display prominent [M+H]⁺ and [M+Na]⁺quasimolecular ions along with fragments corresponding to loss of the neutral sugar cladinose ([M-159]⁺) or mycarose ([M-145]⁺) and a fragment corresponding to desosamine (m/z 158; HRMS gives C₈H₁₆NO₂). These data can be used to tentatively identify the new compounds as shown in Table 2. The identity of the compound eluting at 24.3 min was confirmed as 15-nor-6dEB by co-injection with an authentic sample. The compound eluting at 10.5 min was similarly identified as 15-norerythromycin B based on co-injection with an authentic standard prepared by bioconversion of 15-nor-6dEB using a mutant strain of S. erythraea containing inactivated eryA and eryF genes. The compound eluting at 23.5 min gave identical MS data to 15-norerythromycin B, suggesting it is an isomer of that compound such as 15-nor-6-deoxyerythromycin A. Co-injection with 15-nor-6-deoxyerythromycin A revealed it to be a different, as yet unidentified compound, however. Weber et al. (1991) have reported the production of 15-nor-6-deoxyerythromycins by a strain of S. erythraea in which the eryF gene had been inactivated by an insertion, but 15-norerythromycin B has not been reported as a product of S. erythraea. The combined yield of all erythromycins produced by the TEII mutant was decreased less than 20% from the titer determined for the K41-135 strain.

As shown above, the ery-ORF5 TEII caused a major decrease in the use of acetyl-CoA as a chain starter unit by DEBS, as reflected in the greatly decreased amount of 15-nor-6dEB produced in an ery-ORF5⁺ background vs. that produced in the ery-ORF5 mutant. This observation is consistent with editing of the ACP_{L} in the loading module of DEBS1 to remove acetate selectively and is supported by the qualitative biochemical analysis. In this analysis, the TEII enzyme showed a clear preference for an acetylated ACP_{L} domain. (In contrast, the pikromycin TEII enzyme encoded by pikAV exhibits an approx. 3-fold k_{cat}/Kₘ preference for hydrolysis of the propionyl- vs. acetyl-ACP_{L} derivative of the same didomain protein (Kim et al., 2003).) This editing feature most likely derives from the loading AT's ability to incorporate acetyl-CoA in lieu of the natural propionyl-CoA starter unit. Lack of such editing is the most likely reason for the appearance the 15-norerythromycins in culture extracts of the S. erythraea ery-ORF5 mutant. Detection of 6-deoxyerythromycins in this experiment simply reflects the chance that EryF occasionally is unable to act on a compound before it is glycosylated. (Certain 15-nor[6-deoxy]erythromycins have been seen previously in the extracts of an S. erythraea strain in which eryF was disrupted by insertion of plasmid DNA sequences (Weber et al., 1991), most likely because the insertion blocked expression of the ery-ORF5 gene immediately downstream of eryF.) Interestingly, the in vitro data show that butyryl-ACP_{L} is not an ery-ORF5TEII substrate, even though this same species is hydrolyzed by the PikAV TEII (Kim et al., 2003). Although incorporation of an acetyl starter unit by DEBS certainly occurs in vivo, 6dEB derivatives resulting from utilization of a butyryl-CoA starter unit are not observed. That this is not observed to any significant extent in vivo, is most likely because of insufficient butyryl-CoA.

Others (Heathcote et al., 2001; (Kim et al., 2002) have proposed that the ery-ORF5 and PikAV TEII enzymes may also edit the ACP domains in other modules of DEBS to remove aberrantly loaded or decarboxylated substrates. In Example 3, above, it was also found that the yield of 15-methyl-6dEB was increased 2-fold by co-expression of the ery-ORF5 and DEBS KS1° genes. Thus, in Example 3, editing of the loading module ACP_{L} is irrelevant because the KS1I mutation prevents processing of the substrates loaded onto the loading module. In contrast, co-expression of the ery-ORF5 TEII gene in the E. coli system had no effect on the titer of 15-methyl-6dEB produced by diketide feeding, but did increase the titer of 6dEB made by DEBS (see Example 5, below), suggesting again that the major role of the TEII enzyme is effected on the loading domain ACP. Consequently, the Examples favor the idea that an editing activity of the ery-ORF5 TEII in the initial step of 6dEB biosynthesis in both a heterologous and homologous genetic background is the main function of this enzyme.

### Example 5

Co-expression of ery-ORF5TEII and eryA DEBS genes in E. coli. and production of polyketides.

Consistent with the effect seen in Streptomyces, expression of the ery-ORF5 TEII gene has been shown to increase 6dEB titers two-fold in E. coli (Pfeifer et al, 2002). The results presented above suggesting a role for TEII in editing the loading domain ACP in Streptomyces and S. erythraea led us to examine if a similar effect was detectable in E. coli, in an attempt to understand the observation of Pfeifer et al. (2002). 6dEB production by the DEBS enzymes, and 15-methyl-6dEB production by the DEBS module2, DEBS2 and DEBS3 enzymes, in the presence and absence of ery-ORF5 expression, were analyzed.

The E. coli K207-3 host strain for 6dEB production has been previously described (Murli, 2003). Briefly, this strain has four T7 promoter regulated genes integrated in the chromosome: sfp (required to pantetheinylate the DEBS proteins), prpE (required to convert propionate to propionyl-CoA) and accA1/pccB (required to convert propionyl-CoA to (2S)-methylmalonyl-CoA). Plasmids pKOS207-129 and BP130 expressing the DEBS1 and DEBS2 & -3 proteins, respectively, from T7 promoters have been previously described (Murli, 2003; Pfeifer et al., 2001). Plasmid pKOS207-142a is similar to pKOS207-129 except that the NdeI-SpeI fragment encoding the DEBS1 PKS in pKOS207-129 is replaced by the NdeI-SpeI fragment encoding DEBS1 module 2 only from pRSG64 (Gokhale et al., 1999). To generate an E. coli expression vector for ery-ORF5 that was compatible with the DEBS plasmids, the ery-ORF5 PCR fragment used to generate pKOS146-124B described below (in the “TEII purification and in vitro analysis” section) was cloned as a blunt PCR fragment into pCR-Blunt (Invitrogen Corporation) generating pKOS146-124 and sequenced. The NdeI-NsiI ery-ORF5-encoding fragment was cloned from pKOS146-124 into pKOS116-172a (Dayem et al., 2002) generating pKOS149-159g92. Finally, the ery-ORF5-encoding NdeI-AvrII fragment from pKOS149-159g92 was cloned into the backbone of pKOS207-15a (Murli, 2003) between the T7 promoter and the T7 terminator. The resulting plasmid, pKOS285-93, is a pACYC derivative encoding T7promoter-ery-ORF5-T7 terminator.

For polyketide analysis in E. coli, fresh transformants of production strains carrying the indicated plasmids were grown overnight in LB medium supplemented with the appropriate antibiotics (carb, strep and tet). These cultures were diluted 1:50 into 25 ml of fresh LB medium with tetracycline only in 250 ml shake flasks and grown at 37° C. until the OD₆₀₀ reached 0.4 to 0.5. The cultures were cooled to room temperature (25° C.), induced with 0.5 μM isopropyl-β-D-thiogalactopyranoside (IPTG), and the following media supplements were added: 5 mM sodium propionate, 50 mM succinate and 50 mM monosodium glutamate. When necessary, propyl diketide was fed at a final concentration of 0.5 g l⁻¹. The cultures were grown for an additional 48 hours at 22° C. At the end of the fermentation, the OD₆₀₀ was determined and the cells were collected by centrifugation. Five ml of cell free supernatant was extracted with an equal volume of ethyl acetate. The organic fraction (top layer) was removed and dried under vacuum. The residue was resuspended in 500 μl of methanol. An appropriate dilution was analyzed by LC-MS and quantified by ELSD as previously described (Dayem et al., 2002; Murli, 2003). Polyketides were quantified by comparing the ELSD peak area to a standard curve of peak areas generated from an authentic sample. Polyketide titers are reported as averages with standard errors of duplicate or triplicate samples, determined from independent colonies of the strains analyzed.

Consistent with the previous report (Pfeifer et al, 2002), an approximately two-fold increase in 6dEB titers in E. coli was found when ery-ORF5 was co-expressed (Table 3). However, in the absence of a loading domain, i.e., when 15-methyl-6dEB was produced by diketide feeding to the strain with DEBS module2, DEBS2 and DEBS3, ery-ORF5 co-expression did not increase titers significantly (Table 3). Analyses of protein extracts from these strains showed that ery-ORF5 co-expression did not affect the levels of the DEBS proteins. These observations support the suggested role of the ery-ORF5 TEII in editing the loading domain ACP of DEBS and thereby increasing titers in E. coli. For unknown reasons production of 15-nor-6dEB has not been observed in this host when the eryA DEBS genes are expressed under the growth conditions used in spite of ample acetyl-CoA levels. It is possible that the lack of an effect on 15-methyl-6dEB titers in E. coli is due to differences between the acyl-CoA pools in E. coli and Streptomyces sp. or S. erythraea that lead to less mis-acylation of the ACP domains in the extender modules in E. coli. Another factor may be the absence of the ACP_{L} domain in the E. coli system compared with the DEBS KS1° mutant used for diketide feeding experiments in S. coelicolor.

### References

Aparicio, J. F., Molnar, I., Schwecke, T., Konig, A., Haydock, S. F., Khaw, L. E., Staunton, J. & Leadlay, P. F. (1996). Organization of the biosynthetic gene cluster for rapamycin in Streptomyces hygroscopicus: analysis of the enzymatic domains in the modular polyketide synthase. Gene 169, 9-16.

August, P. R., Tang, L., Yoon, Y. J. & 9 other authors. (1998). Biosynthesis of the ansamycin antibiotic rifamycin: deductions from the molecular analysis of the rif biosynthetic gene cluster of Amycolatopsis mediterranei S699. Chem. Biol. 5, 69-79.

Brunker, P., Minas, W., Kallio, P. T. & Bailey, J. E. (1998). Genetic engineering of an industrial strain of Saccharopolyspora erythraea for stable expression of the Vitreoscilla haemoglobin gene (vhb). Microbiology 144, 2441-2448.

Burg, R. W., Miller, B. M., Baker, E. E. & 12 other authors. (1979). Averrnectins, new family of potent anthelmintic agents: producing organism and fermentation. Antimicrob. Agents Chemother. 15, 361-367.

Butler, A. R., Bate, N. & Cundliffe, E. (1999). Impact of thioesterase activity on tylosin biosynthesis in Streptomyces fradiae. Chem. Biol. 6, 287-292.

Caffrey, P., Bevitt, D. J., Staunton, J. & Leadlay, P. F. (1992). Identification of DEBS1, DEBS2 and DEBS3, the multienzyme polypeptides of the erythromycin-producing polyketide synthase from Saccharopolyspora erythraea. FEBS Lett. 304, 225-228.

Carreras, C., Frykman, S. & Ou, S. (2002). Saccharopolyspora erythraea-catalyzed bioconversion of 6-deoxyerythronolide B analogs for production of novel erythromycins. J. Biotechnol. 92, 217-228.

Coronan, J. E. J. R., Charles 0. (1996). Biosynthesis of member lipids. In Escherichia coli and Salmonella, pp. 629-630. Edited by E. C. C. L. J. L. I. Roy Curtiss III, K. Brooks Low, Boris Magasanik, William S. Reznikoff, Monica Riley, Moselio Scharchter, H. Edwin Umbarger. Washington, D.C.: American Society of Microbiology.

Cortes, J., Haydock, S. F., Roberts, G. A., Bevitt, D. J. & Leadlay, P. F. (1990). An unusually large multifunctional polypeptide in the erythromycin-producing polyketide synthase of Saccharopolyspora erythraea. Nature 348, 176-178.

Dayem, L. C., Carney, J. R., Santi, D. V., Pfeifer, B. A., Khosla, C. & Kealey, J. T. (2002). Metabolic engineering of a methylmalonyl-CoA mutase-epimerase pathway for complex polyketide biosynthesis in Escherichia coli. Biochemistry 41, 5193-5201.

Doi-Katayama, Y., Yoon, Y. J., Choi, C. Y., Yu, T. W., Floss, H. G. & Hutchinson, C. R. (2000). Thioesterases and the premature termination of polyketide chain elongation in rifamycin B biosynthesis by Amycolatopsis mediterranei S699. J. Antibiot (Tokyo) 53, 484-495.

Donadio, S. & Katz, L. (1992). Organization of the enzymatic domains in the multifunctional polyketide synthase involved in erythromycin formation in Saccharopolyspora erythraea. Gene 111, 51-60.

Donadio, S., Staver, M. J., McAlpine, J. B., Swanson, S. J. & Katz, L. (1991). Modular organization of genes required for complex polyketide biosynthesis. Science 252, 675-679.

Egan, R. S. & Martin, J. R. (1970). Structure of lankamycin. J. Am. Chem. Soc. 92, 4129-4130.

Evans, P. D., Cook, S. N., Riggs, P. D. & Noren, C. J. (1995). LITMUS: multipurpose cloning vectors with a novel system for bidirectional in vitro transcription. Biotechniques 19, 130-135.

Gokhale, R. S., Hunziker, D., Cane, D. E. & Khosla, C. (1999). Mechanism and specificity of the terminal thioesterase domain from the erythromycin polyketide synthase. Chem. Biol. 6, 117-125.

Haydock, S. F., Aparicio, J. F., Molnar, I. & 7 other authors. (1995). Divergent sequence motifs correlated with the substrate specificity of (methyl)malonyl-CoA:acyl carrier protein transacylase domains in modular polyketide synthases. FEBS Lett. 374, 246-248.

Heathcote, M. L., Staunton, J. & Leadlay, P. F. (2001). Role of type II thioesterases: evidence for removal of short acyl chains produced by aberrant decarboxylation of chain extender units. Chem. Biol. 8, 207-220.

Hu, Z., Bao, K., Zhou, X., Zhou, Q., Hopwood, D. A., Kieser, T. & Deng, Z. (1994). Repeated polyketide synthase modules involved in the biosynthesis of a heptaene macrolide by Streptomyces sp. FR-008. Mol. Microbiol. 14, 163-172.

Hu, Z., Hopwood, D. A., Hutchinson, C. R. (2003). Enhancing heterologous polyketide production in Streptomyces by exploiting plasmid co-integration. Microbiology, submitted.

Jacobsen, J. R., Hutchinson, C. R., Cane, D. E. & Khosla, C. (1997). Precursor-directed biosynthesis of erythromycin analogs by an engineered polyketide synthase. Science 277, 367-369.

Kao, C. M., Katz, L. & Khosla, C. (1994). Engineered biosynthesis of a complete macrolactone in a heterologous host. Science 265, 509-512.

Kieser, T., Bibb, M. J., Buttner, M. J., Chater, K. F. & Hopwood, D. A. (2000). Practical Streptomyces genetics. John Innes Centre, Norwich Reseach Park, Colney, Norwich NR4 7UH, England.

Kim, B. S., Cropp, T. A., Beck, B. J., Sherman, D. H. & Reynolds, K. A. (2002). Biochemical evidence for an editing role of thioesterase II in the biosynthesis of the polyketide pikromycin. J Biol Chem 277, 48028-48034.

Kohli, R. M., Trauger, J. W., Schwarzer, D., Marahiel, M. A. & Walsh, C. T. (2001). Generality of peptide cyclization catalyzed by isolated thioesterase domains of nonribosomal peptide synthetases. Biochemistry 40, 7099-7108.

Lau, J., Cane, D. E. & Khosla, C. (2000). Substrate specificity of the loading didomain of the erythromycin polyketide synthase. Biochemistry 39, 10514-10520.

Liou, G. F., Lau, J., Cane, D. E. & Khosla, C. (2003). Quantitative analysis of loading and extender acyltransferases of modular polyketide synthases. Biochemistry 42, 200-207.

MacNeil, D. J., Gewain, K. M., Ruby, C. L., Dezeny, G., Gibbons, P. H. & MacNeil, T. (1992). Analysis of Streptomyces avermitilis genes required for avermectin biosynthesis utilizing a novel integration vector. Gene 111, 61-68.

Marsden, A. F., Caffrey, P., Aparicio, J. F., Loughran, M. S., Staunton, J. & Leadlay, P. F. (1994). Stereospecific acyl transfers on the erythromycin-producing polyketide synthase. Science 263, 378-380.

Marsden, A. F., Wilkinson, B., Cortes, J., Dunster, N. J., Staunton, J. & Leadlay, P. F. (1998). Engineering broader specificity into an antibiotic-producing polyketide synthase. Science 279, 199-202.

McDaniel, R., Ebert-Khosla, S., Hopwood, D. A. & Khosla, C. (1993). Engineered biosynthesis of novel polyketides. Science 262, 1546-1550.

Molnar, I., Aparicio, J. F., Haydock, S. F., Khaw, L. E., Schwecke, T., Konig, A., Staunton, J. & Leadlay, P. F. (1996). Organisation of the biosynthetic gene cluster for rapamycin in Streptomyces hygroscopicus: analysis of genes flanking the polyketide synthase. Gene 169, 1-7.

Murli, S., Kennedy, J., Dayem, L. C., Carney, J. R. & Kealey, J. T (2003). Metabolic Engineering of Escherichia coli for Improved 6-Deoxyerythronolide B Production. J. Ind. Microbiol. Biotechnol, submitted.

O'Hagan, D. (1993). The polyketide metabolites. Chichester, West Sussex, PO19 1EB, England: Ellis Horwood limited.

Pacey, M. S., Dirlam, J. P., Geldart, R. W. & 7 other authors. (1998). Novel erythromycins from a recombinant Saccharopolyspora erythraea strain NRRL 2338 pIG1. I. Fermentation, isolation and biological activity. J. Antibiot. (Tokyo) 51, 1029-1034.

Pfeifer, B., Hu, Z., Licari, P. & Khosla, C. (2002). Process and metabolic strategies for improved production of Escherichia coli-derived 6-deoxyerythronolide B. Appl Environ Microbiol 68, 3287-3292.

Pfeifer, B. A., Admiraal, S. J., Gramajo, H., Cane, D. E. & Khosla, C. (2001). Biosynthesis of complex polyketides in a metabolically engineered strain of E. coli. Science 291, 1790-1792.

Pieper, R., Ebert-Khosla, S., Cane, D. & Khosla, C. (1996). Erythromycin biosynthesis: kinetic studies on a fully active modular polyketide synthase using natural and unnatural substrates. Biochemistry 35, 2054-2060.

Quadri, L. E., Weinreb, P. H., Lei, M., Nakano, M. M., Zuber, P. & Walsh, C. T. (1998). Characterization of Sfp, a Bacillus subtilis phosphopantetheinyl transferase for peptidyl carrier protein domains in peptide synthetases. Biochemistry 37, 1585-1595.

Schneider, A. & Marahiel, M. A. (1998). Genetic evidence for a role of thioesterase domains, integrated in or associated-with peptide synthetases, in non-ribosomal peptide biosynthesis in Bacillus subtilis. Arch. Microbiol. 169, 404-410.

Schwarzer, D., Mootz, H. D. & Marahiel, M. A. (2001). Exploring the impact of different thioesterase domains for the design of hybrid peptide synthetases. Chem. Biol. 8, 997-1010.

Schwarzer, D., Mootz, H. D., Linne, U. & Marahiel, M. A. (2002). Regeneration of misprimed nonribosomal peptide synthetases by type II thioesterases. Proc Natl Acad Sci USA 99, 14083-14088.

Schwecke, T., Aparicio, J. F., Molnar, I. & 7 other authors. (1995). The biosynthetic gene cluster for the polyketide immunosuppressant rapamycin. Proc. Natl. Acad. Sci. USA 92, 7839-7843.

Tang, L., Fu, H., Betlach, M. C. & McDaniel, R. (1999). Elucidating the mechanism of chain termination switching in the picromycin/methymycin polyketide synthase. Chem. Biol. 6, 553-558.

Tang, L., Shah, S., Chung, L., Carney, J., Katz, L., Khosla, C. & Julien, B. (2000). Cloning and heterologous expression of the epothilone gene cluster. Science 287, 640-642.

Vicira, J. & Messing, J. (1987). Production of single-stranded plasmid DNA. Methods Enzymol. 153, 3-11.

Weber, J. M., Leung, J. O., Swanson, S. J., Idler, K. B. & McAlpine, J. B. (1991). An erythromycin derivative produced by targeted gene disruption in Saccharopolyspora erythraea. Science 252, 114-117.

Wiesmann, K. E., Cortes, J., Brown, M. J., Cutter, A. L., Staunton, J. & Leadlay, P. F. (1995). Polyketide synthesis in vitro on a modular polyketide synthase. Chem. Biol. 2, 583-589.

Wohlert, S., Lomovskaya, N., Kulowski, K., Fonstein, L., Occi, J. L., Gewain, K. M., MacNeil, D. J. & Hutchinson, C. R. (2001). Insights about the biosynthesis of the avermectin deoxysugar L-oleandrose through heterologous expression of Streptomyces avermitilis deoxysugar genes in Streptomyces lividans. Chem. Biol. 8, 681-700.

Xue, Y., Zhao, L., Liu, H. W. & Sherman, D. H. (1998). A gene cluster for macrolide antibiotic biosynthesis in Streptomyces venezuelae: architecture of metabolic diversity. Proc. Natl. Acad. Sci. USA 95, 12111-12116.

Ziermann, R. & Betlach, M. C. (1999). Recombinant polyketide synthesis in Streptomyces: engineering of improved host strains. Biotechniques 26, 106-110.

TABLE 1
BACTERIAL STRAINS AND PLASMIDS

| Strains and plasmid | Relevant characteristics, genotype | References |
|---|---|---|
| Strains | | |
| E. coli ET12567 | dam, dcm, hsdS, cam^{r}, tet^{r} | (MacNeil et |
| | | al., 1992) |
| E. coli XL1-Blue | recA1 endA1 gyrA96 thi-1 hsdR17 supE44 | Stratagene |
| | relA1 lac [F□proAB lacIqZ□M15 Tn10 (Tet^{r})] | |
| E. coli BL21(DE3) | F-ompT hsdS_{B} (r_{B}- m_{B}-) gal dcm (DE3) | Novagen |
| S. coelicolor CH999 | proA1, argA1, redE60 Δact::ermE | (McDaniel et |
| | | al., 1993) |
| S. coelicolor | DEBS carried by high-copy cointegrate | (Hu, 2003) |
| CH999/pKOS11-26* | plasmid like pSMALL described by Hu et al | |
| | (2003). | |
| S. lividans K4-114 | str-6, Δact::ermE | (Ziermann & |
| | | Betlach, |
| | | 1999) |
| S. erythraea K41-135 | An erythromycin high producer | Kosan |
| | | Biosciences, |
| | | Inc. |
| S. erythraea | ery-ORF5::kan | |
| KOS146-171 | | |
| K207-3 | BL21(DE3) ΔprpRBCD::T7promoter-sfp- | Murli et al., |
| | T7promoter-prpE, panD::panD25A, | 2003) |
| | ygfG::T7promoter-accA1-T7promoter-pccB- | |
| | T7terminatore | |
| Plasmids | | |
| pLitmus 28 | Plasmid vector | (Evans et al., |
| | | 1995) |
| pUC119 | Plasmid vector | (Vieira & |
| | | Messing, |
| | | 1987) |
| pJRJ2 | DEBS (KS1 null) expression-vector based on | (Jacobsen et |
| | pRM5 | al., 1997) |
| pWHM467 | Improved pRM5 derivative that facilitates | (Wohlert et |
| | cloning genes bidirectionally under control of | al., 2001) |
| | the respective actI and actIII promoters | |
| pKOS79-170 | A cosmid carrying ery-ORF5 from Sac. | Volchegursky Y. |
| | erythraea K41-135 | unpublished. |
| pKOS97-49B | An E. coli plasmid vector containg oriT and | Z. Hu |
| | apramycin resistance gene | unpublished |
| pKOS146-83A | HindIII/EcoRI segment of pWHM467 cloned | |
| | into pUC119 | |
| pKOS146-87B | Carrying actII-ORF4 in a HindIII/EcoRI | |
| | fragment | |
| pKOS146-88A | A cos site inserted into the NsiI site of pJRJ2- | |
| | like plasmid (Hu147, unpublished). | |
| pKOS146-103A | pWHM467 derivative carrying ery-ORF5 and | |
| | DEBS genes | |
| pKOS146-109 | pWHM467 derivative carrying ery-ORF5 and | |
| | DEBS (KS1 null) genes | |
| pKOS146-119 | A 3 kb BamHI-Bg1II fragment containing eryF, | |
| | ery-ORF5 and eryG cloned into BamHI site of | |
| | pLitmus28 | |
| pKOS146-129B | A kan gene (from Tn5) inserted into PshAI site | |
| | of ery-ORF5 in pKOS146-119 | |
| pKOS146-129C | E. coli plasmid carrying ery-ORF5 flanking | |
| | region with kan inserted into ery-ORF5 gene. | |
| pKOS146-124B | pET16b expression vector containing the ery- | |
| | ORF5 sequence as a NdeI-BamHI cassette | |
| | expressed with an N-terminal 6xHis tag. | |
| BP130 | pET22b derivative encoding T7promoter- | (Pfeifer et al., |
| | DEBS2- | 2001) |
| | ribosome binding site-DEBS3-T7 terminator | |
| pKOS207-129 | RSF1010 derivative encoding T7promoter- | (Murli et al., |
| | DEBS1-T7terminator | 2003) |
| pKOS207-142a | RSF1010 derivative encoding T7promoter- | |
| | DEBSmodule2-T7terminatore | |
| pKOS164-185 | pACYC184 derivative, Δcat | (Murli et al., |
| | | 2003) |
| pKOS285-93 | pKOS164-185 derivative encoding | |
| | T7promoer-eryORF5-T7terminator | |

TABLE 2
LC-MS data for selected erythromycins produced by 129CM

| retention | | | | Neutral | | |
|---|---|---|---|---|---|---|
| time | [M + H]⁺ | [M + Na]⁺ | [M − cladinose]⁺ | sugar | compound | elemental composition |
| 9.3 | 734 | 756 | 576 | cladinose | erythromycin A | calcd for C₃₇H₆₈NO₁₃: |
| | | | | | | 734.4685; |
| | | | | | | found: 734.4654 |
| 10.5 | 704 | 726 | 546 | cladinose | 15-nor- | calcd for C₃₆H₆₆NO₁₂: |
| | | | | | erythromycin B | 704.4580; |
| | | | | | | found: 704.4565 |
| 17.8 | 718 | 740 | 560 | cladinose | erythromycin B | mass standard |
| 23.5 | 704 | 726 | 546 | cladinose | unknown | calcd for C₃₆H₆₆NO₁₂: |
| | | | | | | 704.4580; |
| | | | | | | found: 704.4554 |
| 24.3 | 688 | 710 | 530 | cladinose | 15-nor-6-deoxy- | calcd for C₃₆H₆₆NO₁₁: |
| | | | | | erythromycin B | 688.4630; |
| | | | | | | found: 688.4591 |

TABLE 3
Effect of eryORF5 TEII expression on
polyketide production in E. coli.^{a}

| | | | 6dEB | 15-methyl- |
|---|---|---|---|---|
| Plasmid#1 | Plasmid#2 | Plasmid#3 | mg l⁻¹ | 6dEB mg l⁻¹ |
| DEBS1 | DEBS2 & −3 | vector only | 18 ± 1 | |
| DEBS1 | DEBS2 & −3 | ery-ORF5 TEII | 41 ± 1 | |
| DEBS module2 | DEBS2 & −3 | vector only | | 30 ± 2 |
| DEBS module2 | DEBS2 & −3 | ery-ORF5 TEII | | 24 ± 1 |

^{a}All plasmids were introduced into the production host K207-3. The DEBS1 gene was expressed from pKOS207-129, the DEBS module2 gene from pKOS207-142a, and the DEBS2 & −3 genes from BP130. The pACYC vector control used was pKOS164-185 and ery-ORF5 TEII gene was expressed from pKOS285-93.

What is claimed is:

1. A host cell comprising a polyketide synthase (PKS) gene and a thioesterase II (TEII) gene, wherein said PKS gene has been modified to prevent utilization of the native starter unit for its expressed PKS, and wherein
(a) the PKS gene and the TEII gene are heterologous to the host cell;
(b) the PKS gene and the TEII gene are endogenous to the host cell; or
(c) the PKS gene is endogenous to the host cell and the TEII gene is heterologous to the host cell.

2. The host cell of claim 1(a) wherein the TEII gene and the PKS gene are cognate genes.

3. The host cell of claim 1 wherein the PKS gene has been modified so that the ketosynthase (KS) catalytic domain of module 1 of the PKS gene product is inactive.

4. The host cell of claim 1 wherein the PKS gene is endogenous and the TEII gene is heterologous.

5. The host cell of claim 1 wherein the PKS gene and the TEII gene are both endogenous.

6. The host cell of claim 1 wherein the PKS gene and the TEII gene are both heterologous.

7. The host cell of claim 1, wherein said host cell is selected from the group consisting of Streptomyces and Escherichia.

8. The host cell of claim 7, wherein said host cell is selected from the group consisting of S. erythraea, S. coelicolor, S. lividans, and E. coli.

9. A host cell comprising a heterologous 6-deoxyerythronolide B synthase (DEBS) gene and a heterologous cognate thioesterase II gene, wherein said DEBS gene has been modified so that the ketosynthase catalytic domain of module 1 of the DEBS gene product is inactive, and wherein the host cell is selected from the group consisting of S. erythraea, S. coelicolor, S. lividans, and E. coli.

10. A method of producing a polyketide, comprising culturing the host cell of claim 1 under conditions such that a polyketide is produced.

11. The method of claim 10, further comprising recovering said polyketide.

12. The method of claim 10, wherein said polyketide synthase is 6-deoxyerythronolide B synthase.

13. A method of producing a polyketide, comprising culturing the host cell of claim 9 under conditions such that a polyketide is produced.

14. The method of claim 13 wherein culturing the host cell comprises supplying exogenous propyl diketide to the host cell.

15. A host cell comprising an endogenous thioesterase (TEII) gene and a heterologous polyketide synthase (PKS) gene, wherein the activity of the product of the endogenous TEII gene has been decreased or eliminated.

16. The host cell of claim 15 wherein the endogenous TEII gene has been modified to render its gene product less active or to eliminate its gene product.

17. A method of producing a polyketide, comprising culturing the host cell of claim 15 under conditions such that a polyketide is produced.

18. The method of claim 17, wherein the host cell is S. erythraea and wherein the polyketide comprises 15-nor-6-deoxyerythronolide B.

19. A host cell comprising a polyketide synthase gene, wherein the activity of the endogenous thioesterase II gene product of said host cell has been decreased or eliminated, and wherein the host cell belongs to the species S. erythraea.

20. A host cell of claim 15 that produces a polyketide that is not produced by the host cell before transfection with the heterologous polyketide synthase gene and decrease or elimination of the endogenous TEII activity.

21. The host cell of claim 20 that produces 15-nor-6-deoxyerythronolide B.
